(19) United States
(12) Patent Application Publication
(10) Pub. No.: US 2024/0122468 A1
(43) Pub. Date: Apr. 18, 2024

(54) METHODS AND SYSTEMS FOR IN-SITU INTRAOCULAR LENS TILT MEASUREMENT

(71) Applicant: Alcon Inc., Fribourg (CH)

(72) Inventors: Shannon Frances Fisherkeller, Fort Worth, TX (US); Victor Manuel Hernandez, Fort Worth, TX (US); Nanhong Lou, Southlake, TX (US); Tobias Jura Rapoport, Berlin (DE); Martin Gründig, Rangsdorf (DE); Armin Soltan Zadi, Arlington, TX (US)

(21) Appl. No.: 18/483,660

(22) Filed: Oct. 10, 2023

Related U.S. Application Data
(60) Provisional application No. 63/379,343, filed on Oct. 13, 2022.

(51) Int. Cl. A61B 3/10; A61B 3/00

(52) U.S. Cl. CPC A61B 3/102; A61B 3/0025; A61B 3/0041; A61B 3/1005

(57) ABSTRACT

Certain aspects of the present disclosure provide for in-situ intraocular lens (IOL) tilt determination and for providing information associated with the angle of IOL tilt intraoperatively. An example method includes imaging an IOL within a patient's eye via an ophthalmic imaging device, in-situ, after implantation of the IOL within the patient's eye; processing the image of the IOL within the patient's eye to determine an angle of tilt of the IOL; and outputting information associated with the angle of IOL tilt to a user interface.

### INTRODUCTION

Aspects of the present disclosure relate to in-situ intraocular lens (IOL) tilt determination and correction. As defined herein, in-situ may refer to a period during an IOL implantation surgery on a patient, in which the patient may be supine.

### BACKGROUND

Presbyopia results from a gradual loss of accommodation of the visual system of the human eye. The loss of accommodation is due to an increase in the modulus of elasticity and growth of the crystalline lens of the eye that is located just behind the iris and the pupil.

In the human eye, a circular ciliary muscle surrounds the crystalline lens. Tiny fibers in the eye called ciliary zonules connect the ciliary muscle to the lens capsule that encloses the crystalline lens. The ciliary zonules pull or release the crystalline lens, thereby causing the curvature of the lens to adjust. Adjustment of the curvature of the crystalline lens results in an adjustment of the eye's focal power to bring objects into focus. For example, when the eyes gaze at an object at a distance, the ciliary muscle relaxes causing the ciliary zonules to become taut, which pulls on the lens to have a flatter curve that is better able to focus incoming light rays from distant objects onto the retina.

In a young eye, accommodation is essentially instantaneous and effortless. As the eye ages, the lens becomes less flexible and elastic, and the ciliary muscle strength decreases. These changes result in the reduction of accommodative amplitude (i.e., loss of accommodation) which causes objects that are close to the eye to appear blurry. The loss of near vision is the hallmark sign of presbyopia in people over the age of, e.g., forty. People that are symptomatic of presbyopia typically have difficulty reading small print, such as that on computer display monitors, restaurant menus, and newspaper advertisements, and may need to hold reading materials at arm's length.

A variety of surgical corrective techniques and devices can be used to treat presbyopia, including, for example, implanting multifocal intraocular lenses (IOLs) and accommodation IOLs in the eye and altering the surface of the cornea through corneal ablation techniques.

In cataract surgery, the implanted IOL may exhibit tilt (interchangeably referred to herein as IOL tilt), which can be defined as the angle between the IOL optical axis and a baseline axis. The baseline axis may be the pupillary axis defined as a line perpendicular to the surface of the cornea of the eye, passing through the center of the pupil.

While implantation of IOLs used to treat presbyopia are discussed herein, it should be understood that this is only one example of a use for IOLs and that IOLs may implanted to treat other conditions, and that the aspects described herein with respect to IOL tilt may apply to any such procedure.

IOL tilt may be caused by uneventful cataract surgery, zonular abnormalities, suture fixation, or haptic-optic asymmetric positioning. In scleral-sutured IOLs, multiple factors, such as IOL haptic positioning, suturing errors, lack of capsular support, scleral tunnel positioning, and haptic breakage, can contribute to IOL tilt.

IOL tilt has the potential to induce astigmatism and higher-order aberrations. In one example, the astigmatism, A, may be given by:

A=[P[1+(sin α)²/(3)]]×(tan α)²

The parameter P is the power in diopters of a thin lens in air in the pupillary plane. The parameter a is the tilt angle of the implanted IOL with respect to the pupillary axis. The tilt angle, α, may include IOL tilt in the horizontal and vertical directions. The angle of IOL tilt in the vertical direction (the angle between the optical axis of the IOL, or IOL axis, and a Y-axis) may be given as γ. The angle of IOL tilt in the horizontal direction may be given as (the angle between the optical axis of the IOL and an X-axis) β.

Conventionally, IOL tilt is determined visually by a surgeon after delivery of the IOL to the patient's eye. Such visual determination of the IOL tilt may be subjective and imprecise. Further, using the naked eye, the minimum discernible degree of tilt is estimated to be around 15 degrees, whereas an acceptable degree of tilt may be smaller than 15 degrees (e.g., the acceptable degree of IOL tilt may be around 5 degrees or less). Accordingly, assessing the tilt visually may be inadequate to determine whether there is an acceptably low degree of tilt. In addition, the visually estimated tilt using the naked eye estimates tilt at only one axis at a time.

Accordingly, techniques are needed for improvements in the field of vision correction and, in particular, for objective IOL tilt determination.

### BRIEF SUMMARY

Certain embodiments provide techniques, apparatus, computer-readable mediums, and systems for objective IOL tilt determination. In addition, the determination of the IOL tilt can be determined in-situ. The embodiments described herein allow for non-contact measurement of the IOL tilt. The objective IOL tilt can be determined with increased resolution and repeatability. The in-situ, image-based tilt measurement can find the tilt across any axis of the IOL and, therefore, can provide a comprehensive tilt measurement. The embodiments described herein can, for example, be applied to any top-down surgical image of the IOL.

In some embodiments, the IOL tilt is determined in-situ using image tracing and an arccosine of the major and minor axis. In some embodiments, the IOL tilt is determined in-situ using a Purkinje imaging tilt measurement technique. In some embodiments, the IOL tilt is determined in-situ using an optical coherence tomography (OCT) imaging technique. In some embodiments, the in-situ IOL tilt measurement technique is selected or switched based on the level of tilt.

Certain embodiments provide for outputting of the in-situ measured IOL tilt on a user interface. In some embodiments, the measured IOL tilt is displayed in-situ on a remote display, on an umbilical display, on an imaging tool, on a surgical tool, and/or on another display. In some embodiments, the measured IOL tilt is displayed in-situ as an augmented reality (AR) overlay over an image, such as over an image of a patient's eye. In some embodiments, the measured IOL tilt is displayed in-situ as a numerical value, as an arrow, or by other visual representative means. In some embodiments, the measured IOL tilt is provided as a degree of the tilt and an orientation of the tilt. In some embodiments, information is displayed indicating additional information. Such additional information may guide a surgeon in assessing the IOL tilt. In some embodiments, the additional information is associated with one or more thresholds related to the degree of the tilt. In some embodiments, the additional information may be provided as text information, audio cues, and/or visual cues.

To the accomplishment of the foregoing and related ends, the one or more aspects comprise the features hereinafter fully described and particularly pointed out in the claims. The following description and the appended drawings set forth in detail certain illustrative features of the one or more aspects. These features are indicative, however, of but a few of the various ways in which the principles of various aspects may be employed.

### BRIEF DESCRIPTION OF THE DRAWINGS

The appended figures depict certain aspects of the one or more embodiments and are therefore not to be considered limiting of the scope of this disclosure.

FIG. 1 illustrates anatomy of the human model eye.

FIG. 2 is a flow diagram of an example method for in-situ IOL tilt measurement, according to certain embodiments.

FIG. 3 is an example sagittal vertical cross-section of the eye of FIG. 1 showing an IOL with vertical tilt.

FIG. 4 illustrates the major axis and minor axis of the example IOL of FIG. 3 with vertical tilt in a top-down view.

FIG. 5 is an example sagittal horizontal cross-section of the eye of FIG. 1 showing an IOL with horizontal tilt.

FIG. 6 illustrates the major axis and minor axis of the example IOL of FIG. 5 with horizontal tilt in a top-down view.

FIG. 7 is an example flow diagram for tilt measurement using image tracing and arccosine, according to certain embodiments.

FIG. 8 illustrates example Purkinje reflections of the example eye of FIG. 1.

FIG. 9 illustrates example OCT image for IOL tilt measurement, according to certain embodiments.

FIG. 10 illustrates an example image of an eye with IOL tilt overlay, according to certain embodiments.

FIGS. 11A-11C illustrate another example image of an eye with IOL tilt overlay, according to certain embodiments.

FIG. 12 illustrates an example system for an in-situ IOL tilt measurement and display, in accordance with certain aspects described herein.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

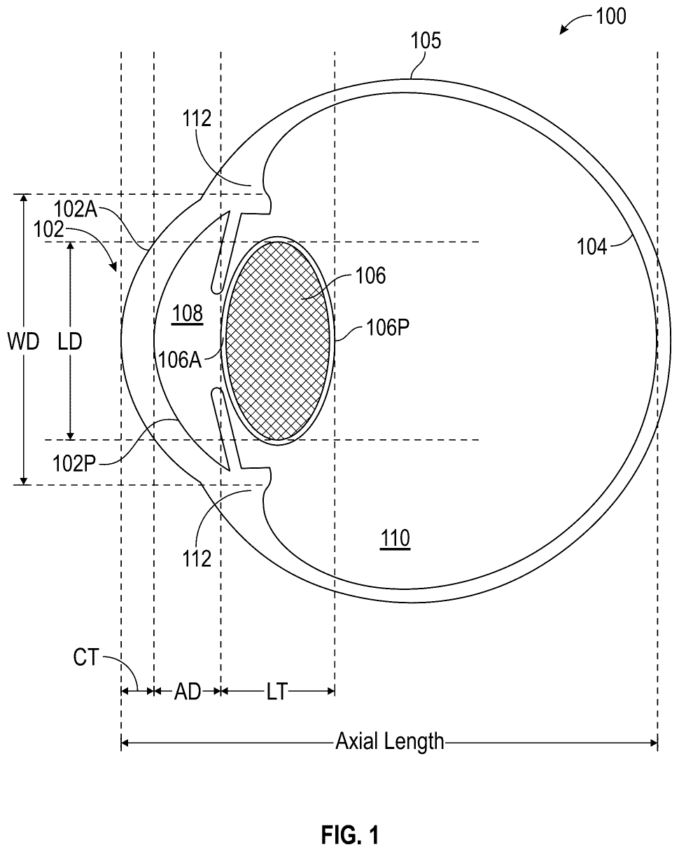

FIG. 1 illustrates anatomy of the human eye 100. As used herein, the anterior side is the side through which light enters the eye, and the posterior side is opposite the anterior side. As shown, the eye 100 includes a cornea 102, a retina 104, a crystalline lens 106, an aqueous humor 108, and a vitreous humor 110. The eye 100 has an overall axial length that is the distance between anterior corneal surface 102A to retina 104.

A thin transparent layer known as the cornea 102 is linked to the sclera 105 by a ring called the limbus, which forms the generally spherical wall of the eye 100. Cornea 102 has a refractive index, n_{cornea}. Cornea 102 has an anterior corneal surface 102A with a radius of curvature, R_{A}, and a posterior corneal surface 102P with a radius of curvature, R_{P}. Cornea 102 has a central corneal thickness (CT) that is the distance between posterior corneal surface 102P and anterior corneal surface 102A.

The iris, the color of the eye, and an opening defined by the iris, the pupil, are positioned behind the cornea and are visible due to the cornea's 102 transparency. The retina 104 is a layer of tissue in the back wall of the eye.

The crystalline lens 106 is a transparent, biconvex structure in the eye 100 that, along with the cornea 102, helps to refract light to be focused on the retina 104. The crystalline lens 106, by changing its shape, functions to change the focal distance of the eye so that the eye can focus on objects at various distances, thus allowing a sharp real image of the object of interest to be formed on the retina 104. This adjustment of the crystalline lens 106 is known as accommodation, and is similar to the focusing of a photographic camera via movement of its lenses. The crystalline lens 106 is positioned behind the iris in a capsular bag. The capsular bag is attached at its equator to the suspensory ciliary muscles 112 by zonule fibers. The ciliary muscles 112 are located beneath the anterior of sclera 105 and can contract or relax in order to change the shape of the crystalline lens 106. Crystalline Lens 106 has a refractive index, nₗₑₙₛ, a lens diameter (LD), and a lens thickness (LT) that is the distance between anterior lens surface 106A and posterior lens surface 106P of crystalline lens 106.

Aqueous humor 108 fills the space between cornea 102 and crystalline lens 106. Aqueous humor 108 has a refractive index, n_{aqueous}. Aqueous humor 108 has an anterior chamber depth (AD) that is the distance between posterior corneal surface 102P apex to the anterior lens surface 106A apex of crystalline lens 106.

Vitreous humor 110 has a depth that is the distance between crystalline lens 106 and retina 104 and a refractive index, nᵥᵢₜᵣₑₒᵤₛ. The anatomy of the human eye 100 also includes a white-to-white diameter (WD) (e.g., the distance between the corneal or scleral boundary on each side of the eye).

Various diseases and disorders of the crystalline lens 106 may be treated with surgery, involving replacing the crystalline lens 106 with an IOL. By way of example, an IOL according to embodiments of the present disclosure may be used to treat cataracts, large optical errors in myopic (near-sighted), hyperopic (far-sighted), and astigmatic eyes, ectopia lentis, aphakia, pseudophakia, and nuclear sclerosis. However, for simplicity, the IOL embodiments of the present disclosure are described with reference to cataracts, which often occur in the elderly population.

In cataract surgery, the crystalline lens 106 is replaced with an IOL. As discussed above, after delivery and unfolding of the IOL, the IOL may be exhibit IOL tilt, which can induce poor optical outcomes. Aspects of the present disclosure provide for in-situ IOL tilt determination. With in-situ IOL tilt determination, the IOL tilt can be measured after implantation of the IOL using objective measurement approaches, while the patient remains supine on the operating table. Aspects provide for displaying the IOL tilt and/or information associated with the IOL tilt on one or more user interfaces (UIs). The IOL tilt information may enable to the surgeon or operator to easily asses the degree of IOL tilt, the direction of the IOL tilt, and/or other information, thereby helping the surgeon or operator to decide whether to take a corrective action with the IOL, such as to adjust the IOL in order to reduce the degree of the IOL tilt to an acceptable degree.

### Example Method of In-Situ Objective IOL Tilt Determination

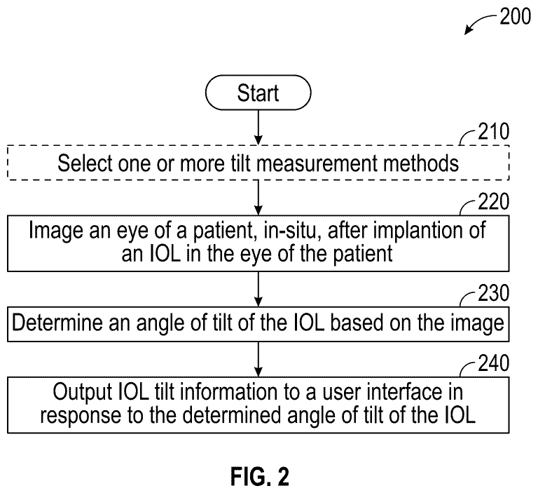

FIG. 2 is a flow diagram of example operations 200 for in-situ IOL tilt measurement, in accordance with certain aspects described herein. In some aspects, operations 200 are performed by one system (e.g., system 1200). In some aspects, operations 200 are performed by multiple systems.

As illustrated, optionally, operations 200 may begin at operation 210, by selecting one or more tilt measurement methods.

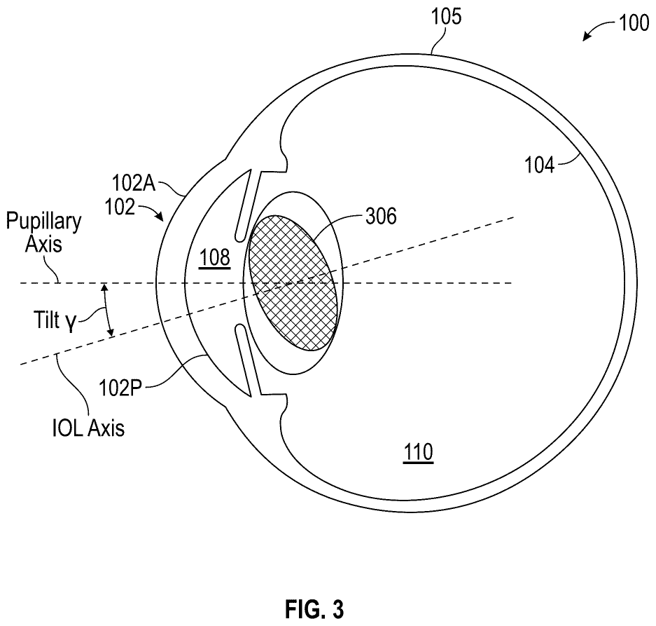

As discussed in more detail below with respect to the FIGS. 3-9, the tilt measurement methods may include an image tracing tilt measurement method, a Purkinje imagining tilt measurement method, or an OCT imaging tilt measurement method. In some examples, multiple (e.g., all three) of the tilt measurement methods can be used simultaneously. In the example of FIG. 2, multiple tilt measurements may be obtained. In some cases, the measurements techniques may be used to determine a final tilt measurement. In some examples, one or more machine learning models may be used to obtain a final tilt measurement from tilt measurements derived from a combination of tilt measurement methods.

In some cases, the different tilt measurement methods may have different effectiveness at different degrees of tilt. For example, the image tracing tilt measurement may be more effective than the Purkinje and OCT imaging tilt measurement methods at high degrees of tilt (e.g., at 15-90 degrees tilt or larger). In some cases, as small degrees of tilt (e.g., 0-10 degrees), the Purkinje imaging tilt measurement technique may provide the most accurate tilt measurement. Accordingly, the tilt measurement method may be selected based on an initial degree of IOL tilt, which can be measured by one of the techniques described herein, estimated visually by a surgeon, or expected based on empirical data. For example, at a large degree of initial tilt (which could be estimated visually by the surgeon or assumed after initial delivery of the IOL), the image tracing tilt measurement method may be used. After adjusting the IOL, or after measurement using the image tracing tilt measurement, the OCT and/or Purkinje imaging tilt measurement may be performed (e.g., alternative to or in addition to the image tracing tilt measurement). When the initial tilt is determined, estimated, or assumed to be small, the Purkinje imaging tilt measurement may be performed (e.g., alternative to or in addition to the OCT and/or image tracing tilt measurement).

Operations 200 continue, at operation 220, with imaging an eye of a patient, in-situ, after implantation of an IOL in the eye of the patient. The imaging of the patient's eye is performed by an ophthalmic imaging device, such as digital surgical microscope, an intra-operative OCT system, an intra-operative aberrometer, or other ophthalmic imaging device. The image of the IOL may be captured, in-situ, by an imaging device used throughout the surgical process and, therefore, may not require any additional imaging devices or repositioning of the patient's eye.

In some embodiments, the imaging device takes a top-down image of the IOL in the patient's eye while the patient is supine. In some embodiments, the imaging device performs a continuous imaging of the IOL in the patient's eye (e.g., to give real-time IOL tilt feedback during delivery, positioning, and/or adjustment of the IOL). In such embodiments, a continuous stream of images may be captured, processed, and provided for visualization by the surgeon. In some embodiments, the continuous (or near continuous) updating may be a real-time video stream of images of the patient's eye with a real-time overlay of the IOL tilt information. In some embodiments, the continuous updating may be done by periodically (e.g., every millisecond, every few milliseconds, every second, every few seconds, etc.) imaging the IOL within the patient's eye, determining the angle of tilt of the IOL, and updating the IOL feedback.

Operations 200 continue, at operation 230, with determining an angle of tilt of the IOL based on the image of the eye of the patient. The angle of tilt of the IOL may be determined based on the selected IOL tilt method. For example, the angle of tilt of the IOL may be determined using image tracing, Purkinje imaging, and/or OCT imaging tilt measurement methods, as discussed in more detail below with respect to the FIGS. 3-9. In some embodiments, the angle of tilt of the IOL is determined by the ophthalmic imaging device. For example, the ophthalmic imaging device has a processor configured to determine the angle of tilt of the IOL (e.g., using image tracing, Purkinje imaging, and/or OCT imaging tilt measurement methods), as discussed in more detail below with respect to FIG. 13. In some other embodiments, the ophthalmic imaging device sends the image of the IOL to a local system or a remote system (e.g., a remote server or cloud) for determining the angle of tilt (e.g., image tracing, Purkinje imaging, and/or OCT tilt measurement imaging methods), as discussed in more detail below with respect to FIG. 13.

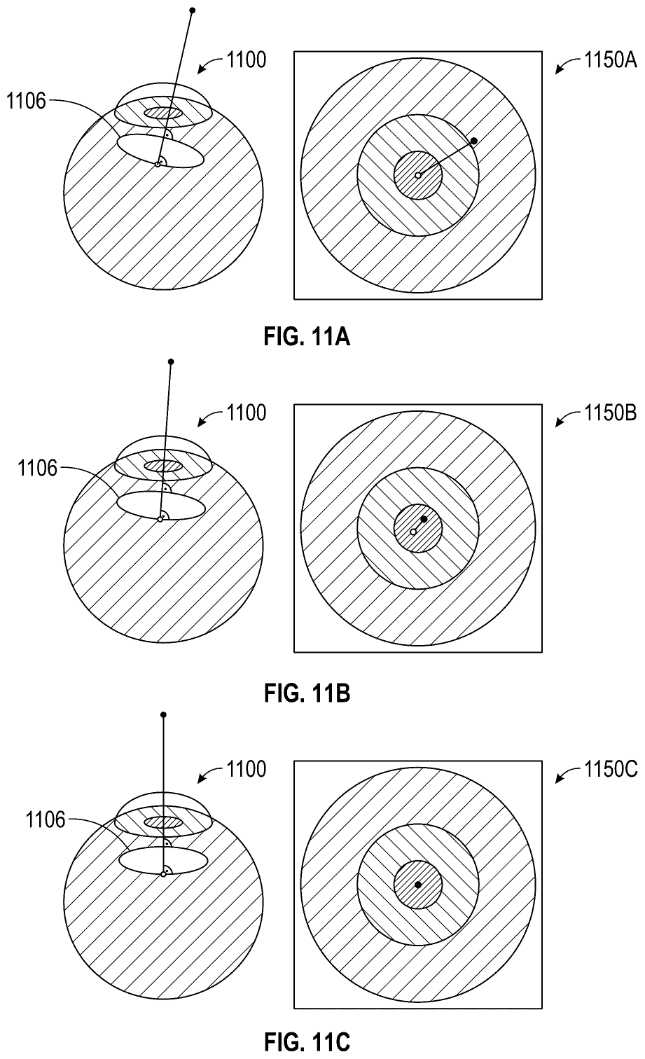

Operations 200 continue, at operation 240, with outputting IOL tilt information to a UI in response to the determining the angle of tilt of the IOL. The IOL tilt information is output to the UI inter-operatively. The IOL tilt information is discussed in more detail below with respect to FIGS. 10-11C. The operator may be the surgeon performing the IOL implantation procedure. In some embodiments, the IOL tilt information is output to a display of the ophthalmic imaging device, an umbilical display coupled to the ophthalmic imaging device, or a remote display, as discussed in more detail below with respect to FIG. 13. The display may provide a user interface for the surgeon.

The surgeon can then use the IOL tilt information to determine whether, and how, to adjust the IOL in the patient's eye, as also discussed in more detail below with respect to FIGS. 10-11C. As discussed above, with continuous IOL tilt feedback, the surgeon can adjust the IOL to correct the IOL tilt based on the IOL tilt feedback and, after adjusting the IOL, the IOL feedback is updated providing the surgeon with information about the angle of IOL tilt after the adjustment, so the surgeon can determine whether the angle of IOL is acceptable or whether (and/or how or how much) to further adjust the IOL to correct the angle of IOL tilt.

### Example In-Situ IOL Tilt Determination

In some embodiments, IOL tilt is determined, at operation 230 of FIG. 2, in-situ, using image tracing. Using an image of the patient's eye, captured at operation 220, the edges of the IOL can be traced.

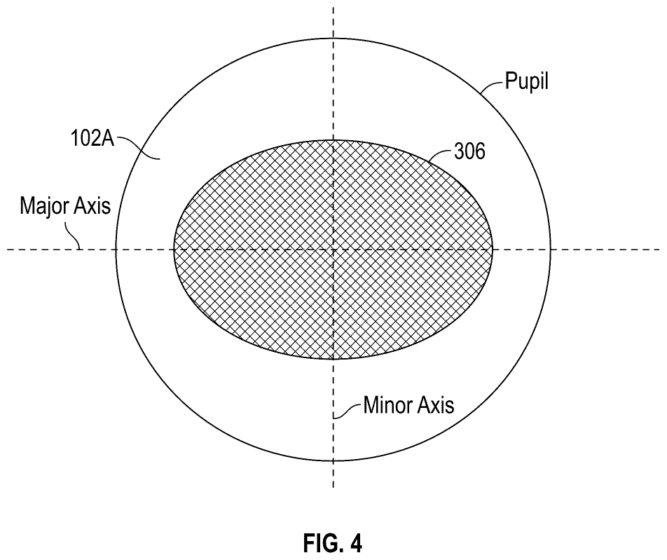

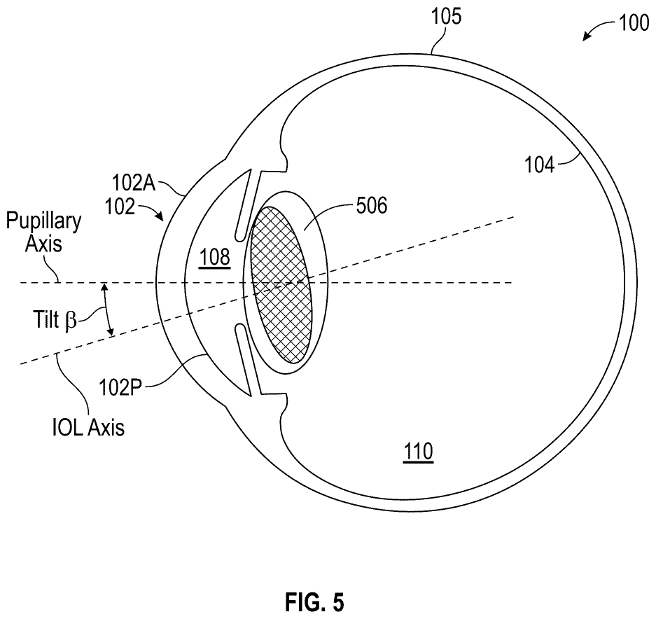

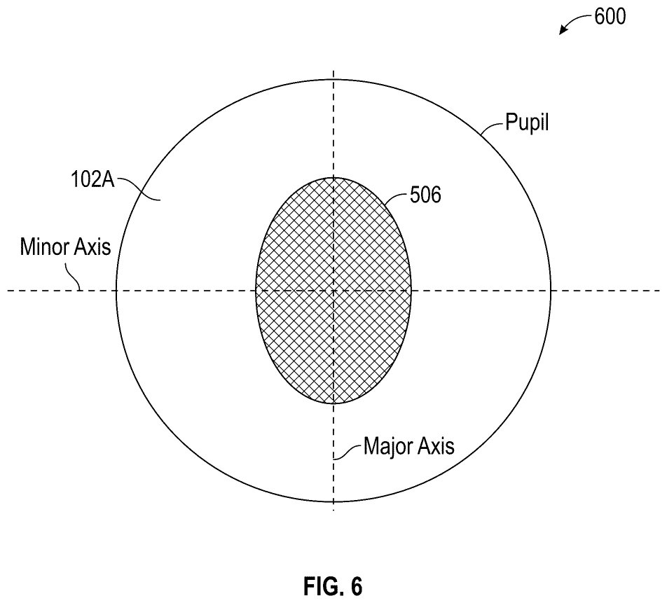

As discussed above, the imaging device may take a top-down image of the IOL that may be used for the image tracing. From the top down view, if the lens were perfectly flat it would appear like a circle. However, if the lens it tilted, it will appear less circular and more like an ellipse. Below, FIGS. 3 and 4 illustrate a vertical tilt of an IOL 306 while FIGS. 5 and 6 illustrate a horizontal tilt of an IOL 506. FIGS. 3 and 5 are cross-sectional views while FIGS. 4 and 6 are top-down views.

FIG. 3 illustrates a sagittal vertical cross-section of the example eye 100 showing an example IOL 306 implanted in the example eye 100 with vertical tilt. The sagittal vertical cross-section is defined by a sagittal plane along the longitudinal axis of a human body. Accordingly, as used herein, vertical tilt refers to tilt in the sagittal plane (i.e., a tilt around the frontal axis of the human body). That is, with vertical tilt, the top and bottom of the IOL 306 tilt in anterior or posterior directions of eye 100, when viewed top-down. As shown, the angle of tilt of IOL 306 in the vertical direction (the angle between the optical axis of the IOL 306, or IOL axis, and the pupillary axis of the eye) may be given as γ. FIG. 4 illustrates an image 400 showing the major axis and minor axis in a top-down view of the example IOL 306 with vertical tilt.

FIG. 5 illustrates a transverse horizontal cross-section of the example eye 100 showing an example IOL 506 implanted in the example eye 100 with horizontal tilt. The transverse horizontal cross-section is defined by a transverse plane perpendicular to the sagittal plane. Accordingly, as used herein, horizontal tilt refers to tilt in the transverse plane around the longitudinal axis of the body. That is, with horizontal tilt, the left and right sides of the IOL 506 tilt in the posterior and anterior directions, when view top-down. As shown, the angle of tilt of IOL 506 in the horizontal direction may be given as (the angle between the optical axis of the IOL 506 and an X-axis) β. FIG. 6 illustrates an image 600 showing the major axis and minor axis in a top-view of the example IOL 506 with horizontal tilt. While FIGS. 3-6 illustrate vertical and horizontal tilt, it should be understood that the IOL may be tilted in both vertical and horizontal directions.

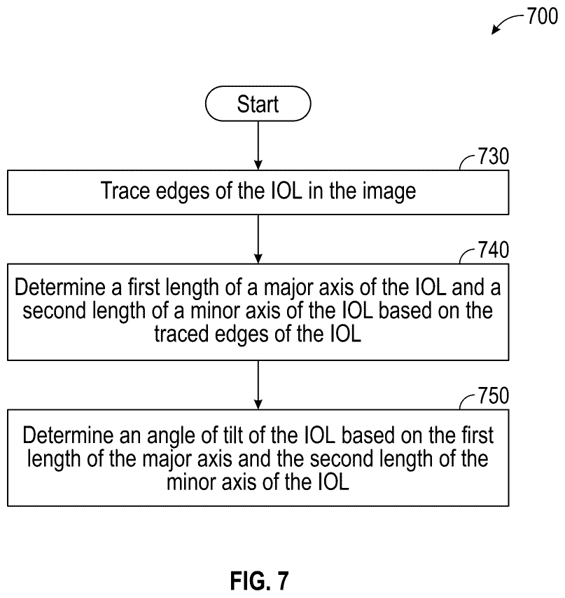

FIG. 7 is an example flow diagram illustrating operations 700 for tilt measurement using image tracing. Operations 700 illustrate the tilt measurement operation 230 of FIG. 2 performed using image tracing.

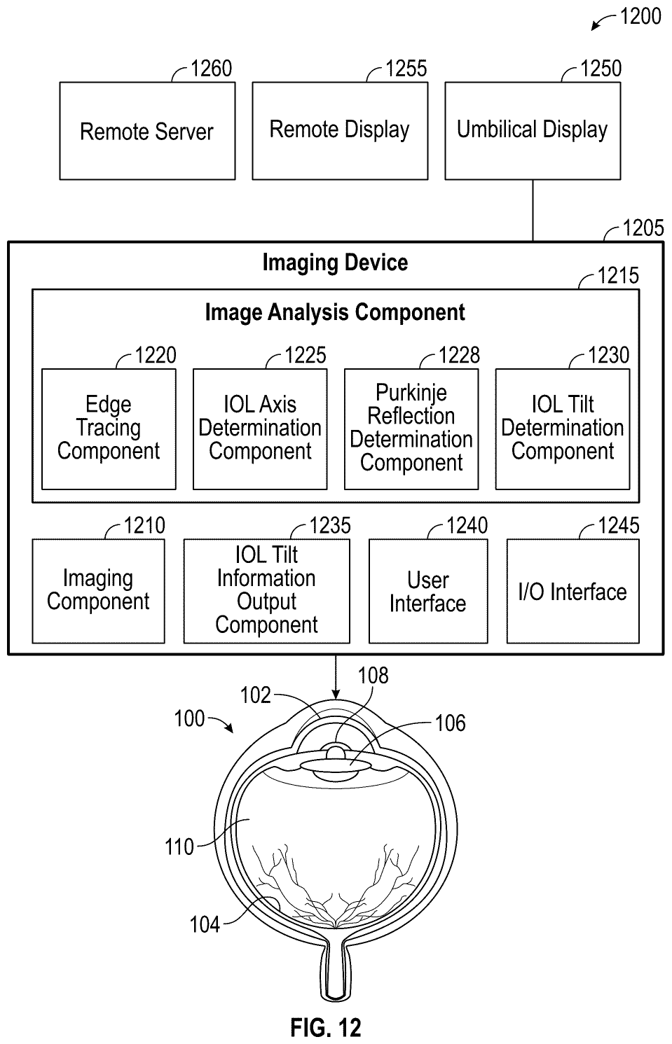

For example, once an IOL is implanted and an image thereof is captured, at operation 710, the edges of the IOL in the image are traced. In some embodiments, the edges of the IOL can be traced using an image recognition and/or analysis (hereinafter referred to “image analysis”) application. Note that an application may also be referred to as a component in FIG. 12. In certain embodiments, the image analysis application executes on a processor of the imaging device and/or another local or remote computing device. The image analysis application may refer to a set of software instructions that may take the image of the IOL as input and (e.g., automatically (e.g., without user input)) provide the traced edges of the IOL in the form of, for example, pixel information associated with pixels that illustrate the edges of the IOL. In certain embodiments, the image analysis application includes a machine learning model that is trained based on a training dataset including a variety of historical IOL images that are labeled with information about the edges of the corresponding IOLs.

In some embodiments, the image of the IOL is input by the operator to the image analysis application. In some embodiments, the IOL image is input automatically by the imaging device to the image analysis application.

At operation 720, a first length of a major axis of the IOL and a second length of a minor axis of the IOL are determined based on the traced edges of the IOL. In some embodiments, the major axis and the minor axis are (e.g., automatically (e.g., without user interaction)) determined by the image analysis application. The minor axis is the axis of greatest tilt. Thus, in the case of vertical tilt, the horizontal axis is the major axis and the vertical axis is the minor axis as shown in FIG. 4 and, in the case of horizontal tilt, the major axis is the vertical axis and the horizontal axis is the minor axis as shown in FIG. 6. In some embodiments, the major and minor axis are determined as a unit of pixel length.

At operation 730, the angle of tilt of the IOL is determined based on the first length of the major axis and the second length of the minor axis of the IOL. In some embodiments, the angle of tilt can be determined as an arccosine of the minor and major axes. In some embodiments, the determination of the angle of tilt is based on the range of tilt being measured. For example, angle of tilt=cos⁻¹(minor axis/major axis) when the anterior surface of the IOL is facing up (i.e., the angle of tilt <90°) and angle of tilt=180−cos⁻¹(minor axis/major axis) when the posterior surface of the IOL is facing up (i.e., the angle of tilt >90°). In some embodiments, the direction of the tilt (e.g., right/left, up/down, or combination thereof) is also determined. In some embodiments, the angle of tilt of the IOL is (e.g., automatically (e.g., without user interaction)) determined by the image analysis application.

In some embodiments, the edge tracing, determination of the major and minor axes, and/or determination of the IOL tilt is performed by the imaging device. In some embodiments, the edge tracing, determination of the major and minor axes, and/or determination of the IOL tilt is performed by a separate computing device. In some embodiments, the edge tracing, determination of the major and minor axes, and/or determination of the IOL tilt is performed at a remote server. For example, the image of the IOL can be sent to the remote server and the remote server can perform the edge tracing, determine the major and minor axes, and/or determine the IOL tilt. The server may then send the result of the analysis (e.g., information about the major and minor axes, and/or the IOL tilt) back for display, as discussed below with respect to FIGS. 10-11C.

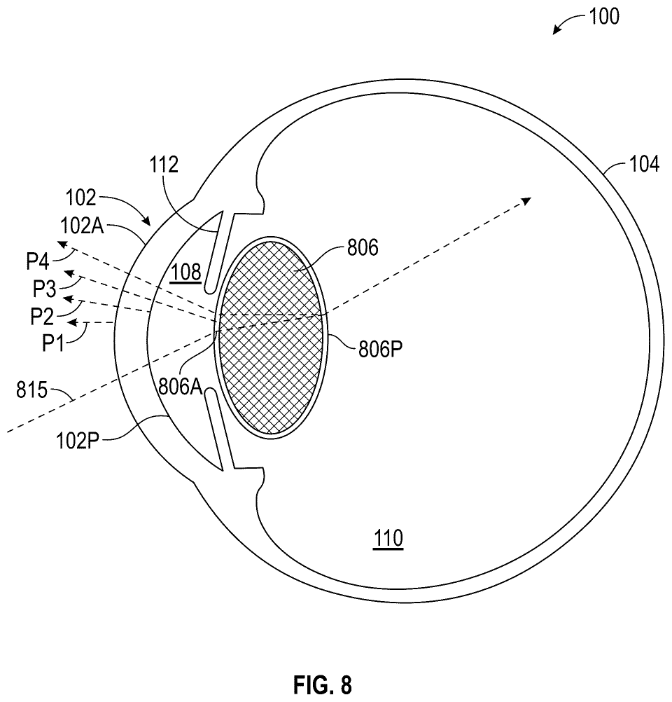

In some embodiments, the IOL tilt is determined, at operation 230, in-situ, using the Purkinje imaging tilt measurement method. When using the Purkinje imaging tilt measurement method, an incident light source, such as the surgical microscope, may be used. FIG. 8 illustrates example Purkinje reflections of the example eye 100 of FIG. 1. As shown, a light ray 815 from the light source propagates into the eye 100 through the pupil and reflects off of various components of the eye 100. For example, with Purkinje imaging, reflections off of the anterior corneal surface 102A (referred to as the first Purkinje image or P1), posterior corneal surface 102P (referred to as the second Purkinje image or P2), anterior IOL surface 806A (referred to as the third Purkinje image or P3), and posterior IOL surface 806P (referred to as the fourth Purkinje image or P4) of the implanted IOL 806 can be measured, as shown in FIG. 8. For a known light source and for no tilt, the positions of the reflections, P1, P2, P3, and P4 are known. When the IOL is tilted, the positions of the reflections will change. Accordingly, the angle (e.g., the magnitude and direction) of IOL tilt can be determined based on the difference between the known positions without tilt and the measured positions with tilt.

In some cases, the Purkinje images, P1 and P4, are formed relatively near to each other within close distance of the pupillary plane approximately in the same plane of focus, while P3 image may be formed in a different plane. Accordingly, the imaging device for use with the Purkinje imaging tilt measurement method may be focused at different planes or a telecentric lens may be used to capture all of the Purkinje images in the same plane. In some embodiments, the determination of the known Purkinje images is based on the anatomical parameters of the eye, such as one or multiple of the anatomical parameters of the eye 100 discussed above with respect to FIG. 1.

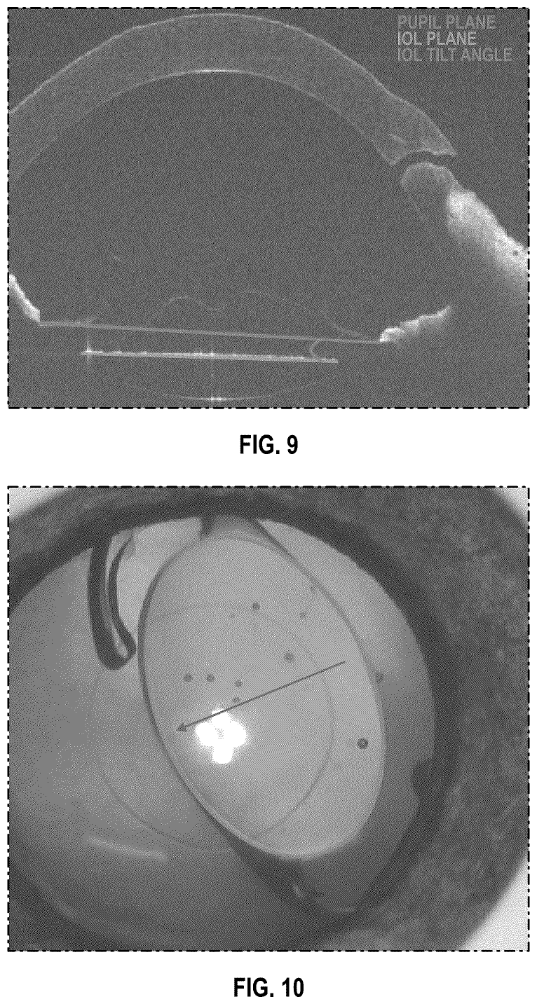

In some embodiments, the IOL tilt is determined, at operation 230 of FIG. 2, in-situ, using OCT imaging tilt measurement. With OCT imaging, cross-sectional information can be obtained from the eye. By scanning at multiple orientations (e.g., by rotating the scan angle, the eye, and/or the microscope), different cross-sections can be obtained about a reference point, such as the apex of the cornea (e.g., the pupillary plane). By comparing the angle of the reference plane and the angle of the IOL relative to the reference plane, the IOL tilt can be determined, as shown in FIG. 9.

### Example IOL Tilt Display

Outputting IOL tilt information, at operation 240 of FIG. 2, to a user interface can provide feedback to a user (e.g., an operator or surgeon) about the angle of IOL tilt.

In some embodiments, the IOL tilt information is an augmented reality (AR) overlay on an image of the patient's eye. FIG. 10 illustrates an example image of an eye with IOL tilt AR overlay. In some embodiments, the IOL tilt information is a numerical value of the IOL tilt, e.g., 35°, as shown in FIG. 10. In some embodiments, the IOL tilt information is represented visually, such as with a line or arrow, also shown in FIG. 10. The line/arrow can have a length representing the magnitude of the angle of IOL tilt and a direction indicating the direction of the IOL tilt, as shown in FIGS. 11A-C. The arrow may represent an artificial optical axis of the lens.

As shown in FIG. 11A, when the IOL 1106 in eye 1100 has large tilt, the image 1150A of the eye 1100 is overlaid with a long axis having a relative magnitude associated with the magnitude of the angle of tilt of the IOL 1106 and a direction indicating the direction of tilt of the IOL 1106. As shown in FIG. 11B, when the IOL 1106 in eye 1100 has medium tilt, the image 1150B of the eye 1100 is overlaid with a medium length arrow, shorter than the arrow in FIG. 11A. As shown in FIG. 11C, when the IOL 1106 in eye 1100 has no tilt, the image 1150C of the eye 1100 is overlaid with no arrow, or a point representing zero tilt of the IOL 1106.

In some embodiments, a color can be used to indicate IOL tilt information. For example, different ranges of IOL tilt may be associated with different colors. For example, zero to five degrees (0°-5°) of IOL tilt may be associated with a color green (e.g., indicating a good/acceptable level of IOL tilt); between five degrees and fifteen degrees (5°-15°) of IOL tilt may be associated with a color yellow (e.g., indicating an intermediate level of IOL tilt); and fifteen degrees (15°) of IOL tilt and beyond may be associated with a red color (e.g., indicating a high/unacceptable level of IOL tilt). The color may be used for the numeric value (e.g., 35° of FIG. 10) and/or for the visual representation (e.g., such as the arrows in FIG. 10 and FIGS. 11A-11C). While three colors associated with three ranges of IOL tilt are described, it should be understood that fewer or more color and associated ranges can be used for providing additional IOL tilt information.

In some embodiments, the IOL tilt information is provided in real-time. For example, the AR overlay may be output to a user interface, such as a display illustrating an image of the eye, and as the IOL is adjusted, the AR overlay may be continuously updated to provide real-time feedback about the IOL tilt. Accordingly, a user (e.g., the surgeon) may use the IOL tilt information as a guide in positioning the IOL in the patient's eye.

In some embodiments, the IOL tilt information is provided via audio means (e.g., an audio output of the numerical value of the IOL tilt).

In some embodiments, the IOL tilt information provides additional information to guide the surgeon to correct the IOL tilt. For example, the IOL tilt information may indicate visually, or via audio, how to correct the IOL tilt. For example, an overlay of the image of the eye may provide a visual cue, such as an arrow, indicating how to adjust the IOL to correct the IOL tilt. For example, in addition to, or alternative to, indicating the angle of IOL tilt and/or the direction of the IOL tilt, an arrow or other visual cue may be used to point in a direction needed to correct the IOL tilt.

In some embodiments, pre-operative measurements of the patient may be used to define patient-specific tilt thresholds. For example, any of the anatomical parameters of the eye 100 discussed above with respect to FIG. 1, or other anatomical parameters of the patient's eye, may be used to determine acceptable ranges of IOL tilt for the patient. In some embodiments, tilt thresholds and/or ranges may be based on a type of the IOL. For example, for multifocal IOLs and extended focus IOLs it may be desirable to achieve a near-zero tilt, while monofocal IOLs may have a higher acceptable tilt threshold. Accordingly, IOL implantation surgeries may have more certainty in the post-operative refractive outcome of the IOL and reduced risk.

Accordingly, the surgeon can use the IOL tilt information to adjust the IOL in order to correct the angle of IOL tilt intra-operatively. For example, after, or while, implanting the IOL within the patient's eye, the surgeon can adjust the position of the IOL to correct the IOL tilt. For example, when the overlay IOL tilt information shows a large angle of IOL tilt (e.g., such as a numerical value above a threshold, a long axis or arrow, and/or the color red indicating an unacceptable level of IOL tilt) the surgeon continues to adjust the IOL until the overlay IOL tilt information shows an acceptable angle of IOL tilt (e.g., such as the numerical value below the threshold, a short axis or arrow, and/or the color green indicating an acceptable level of IOL tilt). In addition, where the overlay information shows a direction of the IOL tilt and/or provides correction guidance information, the surgeon knows how to adjust the IOL to correct the IOL tilt.

### Example System for Designing, Configuring, and/or Forming a Contact Lens with a Smooth Surface Diffractive Design

FIG. 12 illustrates an example system 1200 for IOL tilt determination and IOL tilt information display, in accordance with certain aspects described herein.

As shown, system 1200 may include, but is not limited to, an imaging device 1205, an umbilical display 1250, a remote display 1255, and a remote server 1260.

Imaging device 1205 may be any suitable ophthalmic imaging device, such as an OCT device (e.g., an intraoperative OCT device), a Purkinje imaging tilt measurement system, a digital microscope, scanning laser polarimetry (SLP), a Scheimpflug camera, or other ophthalmic imaging device. The imaging device 1205 may include an imaging component 1210, an image analysis component 1215, an IOL tilt information output component 1235, a user interface 1240, and an input/output (I/O) interface 1245.

The imaging component 1210 may be configured to take an image of an IOL within a patient's eye 100. In some examples, the imaging component 1210 is configured to take a top-image of the patient's eye, in-situ, while the patient is supine. In some embodiments, the imaging component 1210 may be configured to continuously take images of the IOL. In some embodiments, the imaging component 1210 may be configured to image the IOL from different angles, for example, by rotating the eye 100 or the imaging component 1210.

In some embodiments, image analysis component 1215 comprises a control module includes one or more central processing units (CPUs), a memory, and a storage. The CPU may retrieve and execute programming instructions stored in memory. Similarly, the CPU may retrieve and store application data residing in memory. A CPU may have multiple processing cores. The memory may represent a random access memory. Furthermore, in certain aspects, the storage may be a disk drive. The storage may be a combination of fixed or removable storage devices, such as fixed disc drives, removable memory cards or optical storage, network attached storage (NAS), or a storage area-network (SAN).

Image analysis component 1215 may include an edge tracing component 1220, an IOL axis determination component 1225, a Purkinje reflection determination component 1228, and an IOL tilt determination component 1230. Although image analysis component 1215 is shown on image device 1205 in FIG. 12, in some embodiments, image analysis component 1215 may be located on a different device, such as remote server 1260 (e.g., a cloud server). Image analysis component 1215 may be configured to obtain the image of an IOL (e.g., IOL 306, 506, 806, etc.) from the imaging component 1210 and determine the IOL tilt.

In some embodiments, edge tracing component 1220 may be configured to trace the edges of the IOL, as described above, from the image of the IOL obtained from imaging component 1210. IOL axis determination component 1225 may be configured to determine the major and minor axis of the IOL based on the tracing and provide the major and minor axis to the IOL tilt determination component 1230. IOL tilt determination component 1230 may be configured to determine the IOL tilt based on the major and minor axis of the IOL as discussed herein. For example, IOL tilt determination component 1230 may determine the IOL tilt based on an arccosine of the major and minor axis.

In some embodiments, Purkinje reflection determination component 1228 may be configured to determine one or more Purkinje reflections from the image of the IOL obtained from imaging component 1210 and provide information about the Purkinje reflections to the IOL tilt determination component 1230. IOL tilt determination component 1230 may be configured to determine the IOL tilt based on the Purkinje reflections as discussed herein. For example, IOL tilt determination component 1230 may be configured to compare the Purkinje reflections from the IOL to known or expected Purkinje reflection locations and determine the angle of IOL tilt based on differences between the Purkinje reflections from the IOL to the known or expected Purkinje reflection locations.

In some embodiments, IOL tilt determination component 1230 is configured to determine the angle of IOL tilt based on one or more OCT cross-sectional images of the IOL as described herein.

IOL tilt information output component 1235 may be configured to output information associated with the IOL tilt determined by IOL tilt determination component 1230. For example, IOL tilt information component may output any of the information discussed herein, such as a numerical value of the angle of IOL tilt, a visual representation of the IOL tilt, audio information associated with the IOL tilt, an AR overlay, colors associated with ranges of the angle of IOL tilt, and/or guidance information for correction the IOL tilt. In some embodiments, IOL tilt information output component 1235 outputs the information to user interface 1240 on imaging device 1205, to umbilical display 1250, and/or to the remote display 1255.

I/O interface 1245 allows one or more I/O devices (e.g., keyboards, displays, mouse devices, pen input, etc.) to connect to imaging device 1205.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for providing intraocular lens (IOL) tilt measurement intraoperatively, comprising:
imaging an IOL within a patient's eye via an ophthalmic imaging device, in-situ, after implantation of the IOL within the patient's eye to generate an image of the IOL;
processing, by a processor, the image of the IOL to determine an angle of tilt of the IOL, the processing comprising:
automatically tracing edges of the IOL in the image;
automatically determining a length of a major axis of the IOL and a length of a minor axis based on the traced edges of the IOL; and
automatically computing the angle of tilt of the IOL based on the length of the major axis of the IOL and the length of the minor axis of the IOL; and
outputting information associated with the angle of tilt of the IOL to a user interface.

2. The method of claim 1, wherein the imaging of the IOL within the patient's eye, the processing the image of the IOL within the patient's eye, and the outputting the information associated with the angle of tilt of the IOL are performed while the patient is supine.

3. The method of claim 1, further comprising selecting an IOL tilt measurement technique based on an initial degree of tilt, wherein the imaging of the IOL within the patient's eye and/or the processing the image of the IOL within the patient's eye are performed based on the selected IOL tilt measurement technique.

4. The method of claim 1, wherein outputting the information associated with the angle of tilt of the IOL to the user interface comprises overlaying the information associated with the angle of tilt of the IOL on top of the image of the IOL within the patient's eye.

5. The method of claim 1, wherein the imaging of the IOL within the patient's eye and the outputting the information associated with the angle of tilt of the IOL is performed continuously.

6. The method of claim 1, wherein the information associated with the angle of tilt of the IOL comprises one or more of: a numeric value of the angle of tilt of the IOL, a color associated with a range of angle of tilt of the IOL, an artificial optical axis of the IOL, tilt correction guidance information, an audio cue associated with the angle of tilt of the IOL, or a combination thereof.

7. The method of claim 1, wherein:
processing the image of the IOL within the patient's eye to determine the angle of IOL tilt further comprises:
determining a second angle of tilt of the IOL based on one or more optical coherence tomography (OCT) images; and
determining a third angle of tilt of the IOL based on a difference between a first set of measured Purkinje reflections and a second set of expected Purkinje reflections; or
a combination thereof, and
the information associated with the angle of tilt is based on the angle of tilt, the second angle of tilt, and the third angle of tilt.

8. An apparatus for intraoperative intraocular lens (IOL) tilt measurement, comprising:
an imaging component configured to image an IOL within a patient's eye via an ophthalmic imaging device, in-situ, after implantation of the IOL within the patient's eye to generate an image of the IOL;
at least one memory comprising executable instructions; and
at least one processor in data communication with the at least one memory and configured to execute the instructions to:
automatically trace edges of the IOL in the image;
automatically determine a length of a major axis of the IOL and a length of a minor axis based on the traced edges of the IOL; and
compute an angle of tilt of the IOL based on the length of the major axis of the IOL and the length of the minor axis of the IOL; and
output information associated with the angle of tilt of the IOL to a user interface.

9. The apparatus of claim 8, wherein the imaging component is configured to image the IOL within the patient's eye while the patient is supine, and wherein the at least one processor is configured to compute the angle of tilt of the IOL and output the information associated with the angle of tilt of the IOL while the patient is supine.

10. The apparatus of claim 8, wherein the at least one processor is further configured to select an IOL tilt measurement technique based on an initial degree of tilt, and wherein the at least one processor is configured to compute the angle of tilt of the IOL based on the length of the major axis of the IOL and the length of the minor axis of the IOL in response to the selection of the IOL tilt measurement technique.

11. The apparatus of claim 8, wherein the at least one processor is configured to output the information associated with the angle of tilt of the IOL to the user interface by overlaying the information associated with the angle of tilt of the IOL on top of the image of the IOL within the patient's eye.

12. The apparatus of claim 8, wherein the imaging component is configured to continuously image the IOL within the patient's eye, and wherein the at least one processor is configured to continuously output the information associated with the angle of tilt of the IOL.

13. The apparatus of claim 8, wherein the information associated with the angle of tilt of the IOL comprises one or more of: a numeric value of the angle of tilt of the IOL, a color associated with a range of angle of tilt of the IOL, an artificial optical axis of the IOL, tilt correction guidance information, an audio cue associated with the angle of tilt of the IOL, or a combination thereof.

14. The apparatus of claim 8, wherein:
the imaging component is further configured to image the IOL within a patient's eye via optical coherence tomography (OCT) imaging device;
the at least one processor is further configured to determine a second angle of tilt of the IOL based on one or more OCT images; and
the information associated with the angle of tilt is further based on the second angle of tilt.

15. The apparatus of claim 8, wherein the at least one processor is further configured to measure a set of Purkinje reflections and determine a third angle of tilt of the IOL based on a difference between the measured set of Purkinje reflections and a set of expected Purkinje reflections, and wherein the information associated with the angle of tilt is further based on the third angle of tilt.

16. A non-transitory computer readable medium having instructions stored thereon that, when executed by an apparatus, cause the apparatus to perform a method comprising:
imaging an IOL within a patient's eye via an ophthalmic imaging device, in-situ, after implantation of the IOL within the patient's eye to generate an image of the IOL;
processing the image of the IOL to determine an angle of tilt of the IOL, the processing comprising:
automatically tracing edges of the IOL in the image;
automatically determining a length of a major axis of the IOL and a length of a minor axis based on the traced edges of the IOL; and
automatically computing the angle of tilt of the IOL based on the length of the major axis of the IOL and the length of the minor axis of the IOL; and
outputting information associated with the angle of tilt of the IOL to a user interface.

17. The non-transitory computer readable medium of claim 16, wherein the imaging, the computing, and the outputting are performed while the patient is supine.

18. The non-transitory computer readable medium of claim 16, wherein the outputting the information associated with the angle of tilt of the IOL to the user interface includes overlaying the information associated with the angle of tilt of the IOL on top of the image of the IOL within the patient's eye.

19. The non-transitory computer readable medium of claim 16, wherein the imaging comprises continuously imaging the IOL within the patient's eye, and wherein the outputting comprises continuously outputting the information associated with the angle of tilt of the IOL.

20. The non-transitory computer readable medium of claim 16, wherein the information associated with the angle of tilt of the IOL includes one or more of: a numeric value of the angle of tilt of the IOL, a color associated with a range of angle of tilt of the IOL, an artificial optical axis of the IOL, tilt correction guidance information, an audio cue associated with the angle of tilt of the IOL, or a combination thereof.
